(19) United States
(12) Plant Patent Application Publication
(10) Pub. No.: US 2021/0153414 P1
(43) Pub. Date: May 20, 2021

(54) Calibrachoa Plant Name 'Docalaltidab'

(50) ;

(71) Applicant: Dummen Group B.V., De LIER (NL)

(72) Inventor: Arjan KOOT, OEFFELT (NL)

(73) Assignee: Dummen Group B.V., De LIER (NL)

(21) Appl. No.: 16/873,870

(22) Filed: Aug. 4, 2020

(30) Foreign Application Priority Data
Nov. 14, 2019 (QZ) 2019/2970

(51) Int. Cl. A01H 6/82

(52) U.S. Cl. PLT/413

(57) ABSTRACT

A new and distinct cultivar of Calibrachoa plant named ‘Docalaltidab’, characterized by its outwardly spreading, trailing and decumbent plant habit; vigorous growth habit; freely branching habit; early and freely flowering habit; large flowers with purple-colored petals with darker purple-colored centers with star-shaped yellow-colored throats; and good garden performance.

Botanical designation: Calibrachoa parviflora.

Cultivar denomination: ‘DOCALALTIDAB’.

Cross-reference to a related application and statement regarding prior disclosures by Inventor and Applicant:

This application claims priority to a European Community Plant Breeders' Rights application filed on Dec. 12, 2019, application number 2019/2970. There have been no offers for sale anywhere in the world prior to the effective filing date of this Application and no accessibility to one of ordinary skill in the art could have been derived from the printed Plant Breeder's Rights documents.

The Inventor/Applicant asserts that no publications nor advertisements relating to sales, offers for sale or public distribution occurred more than one year prior to the effective filing date of this application. Any information about the claimed plant would have been obtained from a direct or indirect disclosure from the Inventor. Applicant claims a prior art exemption under 35 U.S.C. 102(b)(1) for disclosure and/or sales prior to the filing date but less than one year prior to the effective filing date.

### BACKGROUND OF THE INVENTION

The present invention relates to a new and distinct cultivar of Calibrachoa plant, botanically known as Calibrachoa parviflora and hereinafter referred to by the name ‘Docalaltidab’.

The new Calibrachoa plant is a product of a planned breeding program conducted by the Inventor in Rheinberg, Germany. The objective of the breeding program is to create new freely branching and early-flowering Calibrachoa plants with numerous large and attractive flowers.

The new Calibrachoa plant originated from a cross-pollination made by the Inventor in July, 2017 in Rheinberg, Germany of a proprietary selection of Calibrachoa parviflora identified as code number AA16-092046-003, not patented, as the female, or seed, parent with a proprietary selection of Calibrachoa parviflora identified as code number AA-0576, not patented, as the male, or pollen, parent. The new Calibrachoa plant was discovered and selected by the Inventor as a single flowering plant from within the progeny of the stated cross-pollination in a controlled greenhouse environment in Rheinberg, Germany in May, 2019.

Asexual reproduction of the new Calibrachoa plant by vegetative terminal cuttings in a controlled greenhouse environment in Rheinberg, Germany since June, 2019 has shown that the unique features of this new Calibrachoa plant are stable and reproduced true to type in successive generations.

### SUMMARY OF THE INVENTION

Plants of the new Calibrachoa have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘Docalaltidab’. These characteristics in combination distinguish ‘Docalaltidab’ as a new and distinct Calibrachoa plant:
1. Outwardly spreading, trailing and decumbent plant habit.
2. Vigorous growth habit.
3. Freely branching habit.
4. Early and freely flowering habit.
5. Large flowers with purple-colored petals with darker purple-colored centers with star-shaped yellow-colored throats.
6. Good garden performance.

The new Calibrachoa can be compared to plants of the female parent selection. Plants of the new Calibrachoa differ primarily from plants of the female parent selection in flower color as plants of the new Calibrachoa have a larger star-shaped yellow-colored flower throat than plants of the female parent selection.

The new Calibrachoa can be compared to plants of the male parent selection. Plants of the new Calibrachoa differ primarily from plants of the male parent selection in flower color as plants of the new Calibrachoa have purple-colored petals with darker purple-colored centers with star-shaped yellow-colored throats whereas plants of the male parent selection have violet-colored flowers.

Plants of the new Calibrachoa can be compared to plants of Calibrachoa X hybrida ‘Docalaltib’, disclosed in U.S. Plant Pat. No. 30,345. In side-by-side comparisons, plants of the new Calibrachoa differ primarily from plants of ‘Docalaltib’ in the following characteristics:
1. Plants of the new Calibrachoa are more compact than plants of ‘Docalaltib’.
2. Plants of the new Calibrachoa and ‘Docalaltib’ differ in flower color as plants of the new Calibrachoa have purple-colored petals with darker purple-colored centers with star-shaped yellow-colored throats whereas plants of ‘Docalaltib’ have light violet and purple violet bi-colored flowers.

### BRIEF DESCRIPTION OF THE PHOTOGRAPHS

The accompanying colored photographs illustrate the overall appearance of the new Calibrachoa plant showing the colors as true as it is reasonably possible to obtain in colored reproductions of this type. Colors in the photographs may differ slightly from the color values cited in the detailed botanical description which accurately describe the colors of the new Calibrachoa plant.

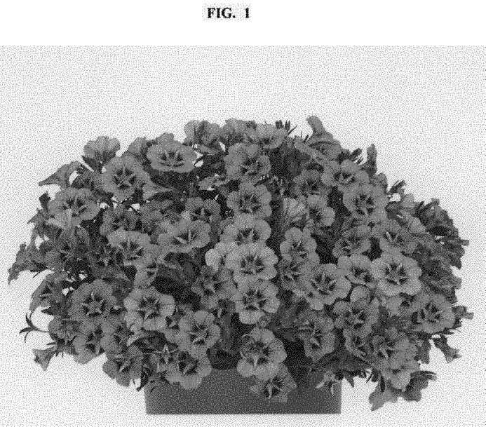

The photograph on the first sheet (FIG. 1 of 2) is a side perspective view of a typical flowering plant of ‘Docalaltidab’ grown in a container.

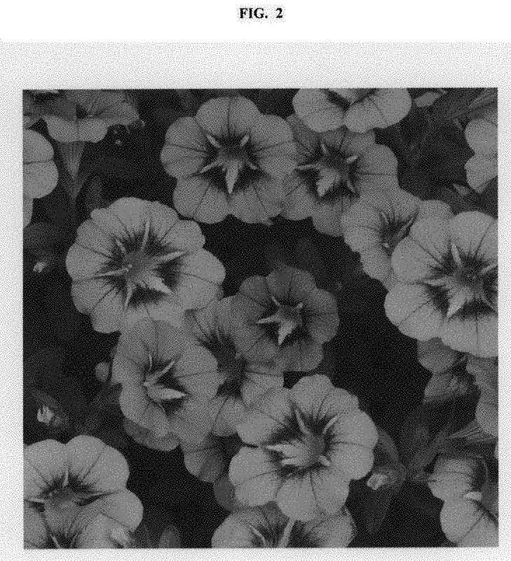

The photograph on the second sheet (FIG. 2 of 2) is a close-up view of typical flowers of ‘Docalaltidab’.

### DETAILED BOTANICAL DESCRIPTION

The aforementioned photographs and following observations, measurements and values describe plants grown during the spring and summer in 22-cm containers in a glass-covered greenhouse in Rheinberg, Germany and under cultural practices typical of commercial Calibrachoa production. During the production of the plants, day and night temperatures averaged 18° C. and light levels averaged 4,500 lux. Rooted young plants were pinched one time three weeks after planting and were twelve weeks old when the photographs and description were taken. In the following description, color references are made to The Royal Horticultural Society Colour Chart, Fifth Edition, 2007, except where general terms of ordinary dictionary significance are used.
Botanical classification: Calibrachoa parviflora ‘Docalaltidab’.
Parentage:
   Female, or seed, parent.—Proprietary selection of Calibrachoa parviflora identified as code number AA16-092046-003, not patented.
   Male, or pollen, parent.—roprietary selection of Calibrachoa parviflora identified as code number AA-0576, not patented.
Propagation:
   Type.—By vegetative cuttings.
   Time to initiate roots, summer.—About five days at temperatures about 20° C.
   Time to initiate roots, winter.—About seven days at temperatures about 20° C.
   Time to produce a rooted young plant, summer.—About three weeks at temperatures about 20° C.
   Time to produce a rooted young plant, winter.—About four weeks at temperatures about 20° C.
   Root description.—Fine, fibrous; close to 158A in color, actual color of the roots is dependent on substrate composition, water quality, fertilizers, substrate temperature and age of roots.
   Rooting habit.—Freely branching; dense.
Plant description:
   Plant and growth habit.—Outwardly spreading to trailing and decumbent plant habit; freely branching habit with about eight primary lateral branches each with about ten secondary lateral branches developing per plant; pinching enhances branching; dense and full appearance; vigorous growth habit; moderate growth rate.
   Plant height, soil level to top of foliar plane.—About 20 cm.
   Plant height, soil level to top of floral plane.—About 20 cm.
   Plant diameter (spread of plant).—About 65 cm.
Lateral branch description:
   Length.—About 45 cm.
   Diameter.—About 5 mm.
   Internode length.—About 2 cm.
   Strength.—Strong.
   Aspect.—Initially upright to outwardly spreading to trailing and decumbent.
   Texture and luster.—Pubescent; glossy.
   Color, developing.—Close to 144B.
   Color, developed.—Close to 152C; at the internodes, close to 144B.
Leaf description:
   Arrangement.—Before flowering, alternate, and after flowering, opposite; simple.
   Length.—About 2.5 cm.
   Width.—About 6.5 mm.
   Shape.—Oblanceolate.
   Apex.—Rounded.
   Base.—Attenuate.
   Margin.—Entire.
   Texture and luster, upper and lower surfaces.—Pubescent; matte.
   Venation pattern.—Pinnate; arcuate.
   Color.—Developing leaves, upper and lower surfaces: Close to 137A. Fully expanded leaves, upper surface: Close to 137C; venation, close to 137C. Fully expanded leaves, lower surface: Close to 137D; venation, close to 137D.
   Petioles.—Length: About 2.5 mm. Diameter: About 1.5 mm. Strength: Moderately strong. Texture and luster, upper and lower surfaces: Pubescent; matte. Color, upper and lower surfaces: Close to 145A.
Flower description:
   Flower arrangement and habit.—Single salverform flowers arising from leaf axils; freely flowering habit with usually about 270 flowers and flower buds per plant; flowers face upright to outwardly.
   Fragrance.—None detected.
   Natural flowering season.—Early flowering habit, plants of the new Calibrachoa initiate and develop flowers about six weeks after planting; plants flower continuously from the spring throughout the summer in Germany.
   Flower longevity.—Individual flowers last about seven to ten days on the plant; flowers not persistent.
   Flower buds.—Length: About 1.7 cm. Diameter: About 3.8 mm. Shape: Elongated oblong. Texture and luster: Rippled; semi-glossy. Color: Close to 82B and 82D.
   Flower diameter.—About 3 cm.
   Flower depth (height).—About 2.7 cm.
   Flower throat diameter.—About 6.5 mm.
   Flower tube length.—About 1.8 cm.
   Flower tube diameter.—About 1.6 mm.
   Corolla.—Arrangement: Five petals fused at the base and opening into a flared trumpet. Petal length from throat: About 1.2 cm. Petal lobe width: About 1 cm. Petal shape: Roughly spatulate. Petal apex: Rounded. Petal margin: Entire; slightly undulate. Petal texture and luster, upper and lower surfaces: Smooth, glabrous; matte. Throat texture: Smooth, glabrous. Tube texture: Smooth, glabrous. Color: Petal, when opening, upper surface: Distally, close to N80A and proximally, close to 83A. Petal, when opening, lower surface: Close to N81C. Petal, fully opened, upper surface: Distally, close to N81B and proximally, close to 83A; venation, close to N81B; colors becoming closer to 86B with development. Petal, fully opened, lower surface: Close to N81C; venation, close to N81C; color becoming closer to 86D with development. Throat: Star-shaped centers, close to 12A; venation, close to 83A. Tube: Close to 4C; venation, close to 83D.
   Calyx.—Arrangement: Star-shaped calyx with five sepals; sepals fused at the base. Sepal length: About 9 mm. Sepal width: About 2.5 mm. Sepal shape: Lanceolate. Sepal apex: Acute. Sepal margin: Entire. Sepal texture and luster, upper and lower surfaces: Pubescent; matte. Color: When developing and fully developed, upper surface: Close to 137C. When developing and fully developed, lower surface: Close to 137D.
   Peduncles.—Length: About 1.2 cm. Diameter: About 1 mm. Angle: About 45° from stem axis. Strength: Moderately strong. Texture and luster: Pubescent; matte. Color: Close to 144B.
   Reproductive organs.—Stamens: Quantity: Five per flower. Filament length: About 7.4 mm. Filament color: Close to 145C. Anther length: About 1 mm. Anther shape: Ellipsoidal. Anther color: Close to 13C. Pollen amount: Scarce. Pollen color: Close to 13A. Pistils: Quantity: One per flower. Pistil length: About 8.5 mm. Style length: About 8 mm. Style color: Close to 149B. Stigma diameter: About 0.5 mm. Stigma shape: Ellipsoidal. Stigma color: Close to 144B. Ovary color: Close to 144C. Fruits: Quantity produced per plant: About 300 per plant. Length: About 6 mm. Diameter: About 4 mm. Texture: Smooth, glabrous. Color: Close to 199C. Seeds: Quantity per flower: About 40. Length: About 1 mm. Diameter: About 1 mm. Texture: Smooth, glabrous. Color: Close to 200A.
Garden performance: Plants of the new Calibrachoa have been observed to have good garden performance and to tolerate wind, rain, temperatures ranging from about 5° C. to about 40° C. and to be suitable for USDA Hardiness Zone 11.
Pathogen & pest resistance: To date, plants of the new Calibrachoa have not been observed to be resistant to pathogens and pests common to Calibrachoa plants.

It is claimed:

1. A new and distinct Calibrachoa plant named ‘Docalaltidab’ as illustrated and described.
